(19) United States
(12) Patent Application Publication
(10) Pub. No.: US 2026/0295191 A1
(43) Pub. Date: Oct. 1, 2026

(54) MEDICAMENT DELIVERY SYSTEM

(71) Applicant: FISHER & PAYKEL HEALTHCARE LIMITED, Auckland (NZ)

(72) Inventor: Stanislav TATKOV, Auckland (NZ)

(21) Appl. No.: 19/565,897

(22) Filed: Mar. 13, 2026

Related U.S. Application Data
(60) Provisional application No. 63/772,241, filed on Mar. 14, 2025.

(51) Int. Cl. A61M 16/06; A61M 11/00

(52) U.S. Cl. CPC A61M 16/0672; A61M 11/00; A61M 16/0683; A61M 2210/0618

(57) ABSTRACT

A medicament delivery system comprising a nasal interface configured to deliver an asymmetric flow of gases to the nares of a patient. The system comprises a nebuliser configured to deliver a nebulised substance into the nasal interface. The nebulised substance is configured to mix with ambient air to form at least part of the gases flow delivered to the nasal interface.

### TECHNICAL FIELD

The present invention relates to a medicament delivery system with a patient interface, and methods of using the system.

### BACKGROUND

A nebuliser turns liquid medicine into a fine mist which can be inhaled into the lungs. A nebuliser may be connected to a patient interface where the nebulised medication is delivered to the patient interface and inhaled by a patient. In some uses, a nebuliser may be connected to the chamber outlet end of a nasal high flow (NHF) device, where medication is dispensed directly into the flow of the breathing gas, and this mixture of breathing gas and nebulised medication travels along the tube and is delivered to the patient via a patient interface.

For various reasons, a full dose of nebulised medication may not be delivered to a patient. This may be undesirable as the patient does not absorb the full intended or expected dose of nebulised medication and also because some medications are expensive. Therefore, manufacturers seek to develop medicament delivery systems that effectively deliver nebulised medication to a patient.

### SUMMARY

In accordance with certain features, aspects and advantages of at least one of the embodiments disclosed herein, there is provided a medicament delivery system comprising: a nasal interface configured to deliver an asymmetric flow of gases to the nares of a patient, the nasal interface comprising a gases inlet; a nebuliser comprising a medicament inlet port and in fluid communication directly or indirectly with the gases inlet, the nebuliser configured to deliver a nebulised substance into the nasal interface; the system defining a flow path between the medicament inlet port and the nasal interface via the gases inlet, wherein the nebulised substance is configured to mix with ambient air to form at least part of the gases flow delivered to the nasal interface.

In some configurations, the nasal interface is non-sealing. A non-sealing interface may have at least one nasal delivery element that is non-sealing.

In some configurations, a medicament delivery chamber is positioned between and directly or indirectly in fluid communication with the gases inlet and the medicament inlet port.

In some configurations, the medicament delivery chamber comprises an inlet for the ambient air and the medicament inlet port.

In some configurations, system comprises a one-way valve upstream of the ambient air inlet to prevent the nebulised substance flowing in a direction through the air inlet.

In some configurations, the medicament delivery chamber comprises an oxygen intake port for receiving oxygen from an oxygen source, the oxygen configured to mix with ambient air in the medicament delivery chamber to form at least part of the gases flow.

In some configurations, the oxygen is provided at a flow rate of approximately 15 L/min to 95 L/min, optionally 25 L/min to 80 L/min, optionally between 35 L/min and 70 L/min.

In some configurations, the oxygen is provided at a flow rate of approximately 3 L/min to 25 L/min. This may be beneficial in some home-use situations.

In some configurations, the system comprises a flow delivery device in fluid communication with the air inlet and positioned upstream of the air inlet to drive the gases flow in the flow path direction toward the nasal interface to increase a gases flow rate.

In some configurations, the system comprises a flow restrictor in fluid communication with the air inlet port and positioned upstream of the air inlet port to restrict a gases flow rate through the nasal interface.

In some configurations, the nasal interface comprises an inlet conduit, wherein the inlet conduit is the medicament delivery chamber.

In some configurations, the air inlet and medicament inlet port are combined into a common port.

In some configurations, the stopping of delivery or reduction of delivery rate of the nebulised substance is at the final 150 ml of the inhalation.

In some configurations, a forced air or oxygen supply is connected upstream to the nebuliser, the forced supply driving a flow through the nebuliser.

In some configurations, the oxygen is provided from an oxygen tank.

In some configurations, the nebuliser is configured to nebulise a liquid medication supplied to the nebuliser to form the nebulised substance.

In some configurations, the medicament delivery chamber (or nebuliser) is directly coupled to the inlet or an inlet tube or conduit of the nasal interface.

In some configurations, nebulised drugs are drawn into the nasal interface due to the asymmetric flow and negative pressure created due to the asymmetric nasal delivery element (e.g. prongs).

In some configurations, the asymmetric flow is configured to increase the inhalation pressure formed in the nasal interface to deliver the nebulised substance mixed with ambient air to the user.

In some configurations, the nasal interface comprises first and second nasal delivery elements configured to deliver the asymmetric flow of gases to the nares.

In some configurations, the first nasal delivery element is larger than the second nasal delivery element.

In some configurations, the first nasal delivery element has a larger internal diameter and/or internal cross-sectional area and a larger circumference or external diameter at or adjacent the first nasal delivery element outlet than the second nasal delivery element.

In some configurations, the first delivery element has a diameter or major axis that is larger than the second delivery element.

In some configurations, the first and second nasal delivery elements are asymmetrically sized.

In some configurations, the first nasal delivery element has a larger internal cross-sectional area than the second nasal delivery element.

In some configurations, the first nasal delivery element is relatively larger and the second nasal delivery element is relatively smaller to provide a leak path for expired gases at the patient's other naris.

In accordance with further certain features, aspects and advantages of at least one of the embodiments disclosed herein, there is provided a respiratory therapy system comprising: a liquid medication source; a gas delivery apparatus configured to provide respiratory therapy to a patient, and a medicament delivery system comprising: a nasal interface configured to deliver an asymmetric flow of gases to the nares of a patient, the nasal interface comprising a gases inlet; a nebuliser and/or a medicant delivery chamber directly or indirectly in fluid communication with the liquid medication source and the gases inlet, the nebuliser configured to nebulise liquid medication from the liquid medication source and deliver resultant nebulised medication to the nasal interface, wherein the nebulised medication is configured to form at least part of a gases flow delivered to the nasal interface.

In some configurations, the medicament delivery system is as hereinbefore described.

In accordance with further certain features, aspects and advantages of at least one of the embodiments disclosed herein, there is provided a method of operating a respiratory therapy system, the method comprising steps of: controlling a flow generator of the system to generate a flow of ambient air to the medicament delivery system; controlling a nebuliser to deliver nebulised medication to the medicament delivery system; controlling the flow generator such that a gases flow of ambient air and nebulised medication is delivered to the patient via the nasal interface; wherein the gases flow is delivered to the patient's nares asymmetrically.

In some configurations, the respiratory therapy system is as hereinbefore described.

In some configurations, the method comprises stopping respiratory therapy when nebulising is required.

In some configurations, wherein the flow generator is reduced to a relatively low flow to prevent humidity flowing back toward the flow generator. Preferably, a relatively low flow is less than about 10 L/min. More preferably, a relatively low flow is less than about 5 L/min. Even more preferably, a relatively low flow is less than about 3 L/min.

In some configurations, the medicament delivery system comprises a cannula removably connected between the nasal interface and the gas delivery apparatus, wherein the method comprises disconnecting the cannula from the gas delivery apparatus and connecting the nebuliser directly to the cannula when nebulising is required.

In some configurations, the gas delivery apparatus provides a constant flow rate between 1 L/min to 5 L/min as a standby flow when respiratory therapy is not required.

In some configurations, a humidity output of the gas delivery apparatus is reduced when respiratory therapy is not required.

In accordance with further certain features, aspects and advantages of at least one of the embodiments disclosed herein, there is provided a respiratory support system comprising: a nasal interface comprising a gases inlet for a flow of gases through the nasal interface, and configured to deliver an asymmetric flow of gases to the nares of a patient, a nebuliser configured to deliver nebulised medication, the nebuliser fluidly coupled to the gases inlet of the nasal interface, wherein the flow of gases through the nasal interface is adapted to create a negative pressure sufficient to draw the nebulised medication through the nasal interface.

In some configurations, the first nasal delivery element and second nasal delivery element are asymmetric.

In some configurations, the first nasal delivery element occludes the first naris more than the second nasal delivery element occludes the second naris.

In some configurations, the nasal interface comprises asymmetric nasal delivery elements, wherein a first nasal delivery element is larger than the second nasal delivery element, and the first nasal delivery element is configured to occlude at least 25% more area of the naris than the second nasal delivery element.

In some configurations, the respiratory therapy system comprises a blood oxygen saturation sensor; an oxygen inlet; and a valve in fluid communication with the oxygen inlet to control a flow of oxygen through the oxygen inlet; wherein the controller is configured to control the valve based on at least one measurement of oxygen saturation from the blood oxygen saturation sensor.

In accordance with further certain features, aspects and advantages of at least one of the embodiments disclosed herein, there is provided a method of providing nebulised medication, the method comprising steps of: providing a non-sealing nasal interface for delivering an asymmetric flow of gases to the nares of a patient, connecting a nebuliser and/or a medicant delivery chamber directly or indirectly in fluid communication with a liquid medication source and the nasal interface, delivering a nebulised medication to the nasal interface from the liquid medication source, wherein the nebulised medication is drawn by the asymmetric flow at the nares of the patient.

In some configurations, the method comprises providing respiratory therapy via the nasal interface.

In some configurations, the method comprises stopping respiratory therapy when nebulising is required.

In some configurations, the method comprises nebulising liquid medication from the liquid medication source and delivering the resultant nebulised medication to the nasal interface.

In some configurations, the asymmetric flow is provided by asymmetric sized nasal delivery elements at the patient's nares, wherein one nasal delivery element occludes an associated naris more than the other nasal delivery element, the occlusion of the naris creating negative pressure during inspiration to draw the nebulised medication.

In some configurations, the method comprises delivering ambient air and mixing the nebulised medication with the ambient air and delivering to the nasal interface.

In some configurations, the method comprises controlling a flow generator to generate a flow of ambient air and/or oxygen to the nasal interface.

In some configurations, the flow generator is in fluid communication with the nebuliser and/or the medicant delivery chamber.

In some configurations, the method comprises connecting the nebuliser and/or medicant delivery chamber directly to an inlet or an inlet tube of the nasal interface.

In some configurations, the method comprises comprising providing a leak path at at least one of the patient's naris for the flow of gases.

In accordance with further certain features, aspects and advantages of at least one of the embodiments disclosed herein, there is provided a method of providing respiratory therapy and nebulised medication, the method comprising: providing a non-sealing nasal interface for delivering an asymmetric flow of gases to the nares of a patient, switching between: a first configuration of connecting a flow generator to a gases inlet or gases inlet tube of the nasal interface, the flow generator controlled to deliver a flow of ambient air and/or oxygen to the nasal interface; and a second configuration of connecting a nebuliser and/or a medicant delivery chamber directly or indirectly in fluid communication with a liquid medication source and the nasal interface; wherein in the second configuration, the nebulised medication is drawn into the nares by the asymmetric flow at the nares of the patient.

In some configurations, in switching between the first configuration and second configuration, the method comprises disconnecting the flow generator from the nasal interface.

In some configurations, the method comprises, when in the second configuration, the nebuliser and/or a medicant delivery chamber directly connected to the gases inlet or gases inlet tube.

In some configurations, the method comprises when switching between the first configuration and second configuration, the flow generator remains connected to the nasal interface, and the flow generator is controlled to provide a relatively low level of flow such that the nebulised medication continues to be drawn into the nares by the asymmetric flow at the nares of the patient.

In some configurations, the method comprises the flow generator is in fluid communication with the nebuliser and/or the medicant delivery chamber.

In some configurations, the method comprises providing a leak path at at least one of the patient's naris for the flow of gases.

In some configurations, the method comprises the asymmetric flow provided by asymmetric sized nasal delivery elements at the patient's nares, wherein one nasal delivery element occludes an associated naris more than the other nasal delivery element, the occlusion of the naris creating negative pressure during inspiration to draw the nebulised medication.

In some configurations, the method comprises nebulising liquid medication from the liquid medication source and delivering the resultant nebulised medication to the nasal interface.

In accordance with further certain features, aspects and advantages of at least one of the embodiments disclosed herein, there is provided a respiratory therapy system, the system comprising: a liquid medication source; a gas delivery apparatus comprising a flow generator configured to provide at least part of a gases flow delivered to the nasal interface; a non-sealing nasal interface configured to deliver an asymmetric flow of gases to the nares of a patient; a nebuliser and/or a medicant delivery chamber directly or indirectly in fluid communication with the liquid medication source and the nasal interface, the nebuliser configured to nebulise liquid medication from the liquid medication source and deliver resultant nebulised medication to the nasal interface, wherein the nebulised medication is configured to form at least part of the gases flow delivered to the nasal interface.

In some configurations, the nasal interface comprises a gases inlet for receiving the gases flow, the nebuliser in fluid communication with the liquid medication source and the gases inlet.

In some configurations, the gases flow delivered by the flow generator comprises ambient air and/or oxygen.

In some configurations, the gases flow delivered by the flow generator is a high flow.

In some configurations, the nasal interface comprises first and second nasal delivery elements configured to deliver the asymmetric flow of gases to the nares.

In some configurations, the first nasal delivery element and second nasal delivery element are asymmetric.

In some configurations, the first nasal delivery element occludes the first naris more than the second nasal delivery element occludes the second naris.

In some configurations, the first nasal delivery element is larger than the second nasal delivery element.

In some configurations, the system comprises a delivery tube positioned between the flow generator and the nasal interface for delivering the gases flow between the flow generator and the nasal interface.

In some configurations, the system comprises a controller configured to operate the flow generator according to a detection of a nebulising function by the nebuliser.

In some configurations, the system comprises a user interface configured to selectively operate a nebulising function by the nebuliser.

In accordance with further certain features, aspects and advantages of at least one of the embodiments disclosed herein, there is provided a method of providing respiratory therapy and nebulised medication, the method comprising: providing a non-sealing nasal interface for delivering an asymmetric flow of gases to the nares of a patient, delivering at least part of a gases flow to the nasal interface from a flow generator via a delivery tube; and selectively providing nebulised medication and delivering nebulised medication to form at least part of the gases flow provided to the nasal interface, the nebulised medication provided via a nebuliser.

In some configurations, the method comprises providing high flow respiratory therapy via the nasal interface.

In some configurations, the asymmetric flow is provided by asymmetric nasal delivery elements at the patient's nares, wherein one nasal delivery element occludes an associated naris more than the other nasal delivery element.

In some configurations, the method comprises providing a flow of ambient air and/or oxygen from the flow generator as part of the flow of gases.

In some configurations, the method comprises mixing the nebulised medication with the ambient air and/or oxygen and delivering to the nasal interface as part of the gases flow.

In some configurations, the method comprises nebulising liquid medication from a liquid medication source and delivering the resultant nebulised medication to the nasal interface.

In some configurations, the method comprises reducing a flow rate of the gases flow from the flow generator when nebulised medication is delivered.

In some configurations, the flow generator detects when nebulised medication is delivered.

In some configurations of any of the nasal interfaces described herein, the nasal interface is configured to provide pressure and asymmetrical flow at the patient's airways in use.

Features from one or more embodiments or configurations may be combined with features of one or more other embodiments or configurations. Additionally, more than one embodiment or configuration may be used together in a respiratory support system during a process of respiratory support of a patient.

As used herein the term “(s)” following a noun means the plural and/or singular form of that noun.

As used herein the term “and/or” means “and” or “or”, or where the context allows both.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

This disclosure may also be said broadly to consist in the parts, elements and features referred to or indicated in the specification of the application, individually or collectively, and any or all combinations of any two or more said parts, elements or features, and where specific integers are mentioned herein which have known equivalents in the art to which this disclosure relates, such known equivalents are deemed to be incorporated herein as if individually set forth.

The disclosure consists in the foregoing and also envisages constructions of which the following gives examples only.

### BRIEF DESCRIPTION OF THE DRAWINGS

Specific embodiments and modifications thereof will become apparent to those skilled in the art from the detailed description herein having reference to the figures that follow, of which:

FIG. 1 is a perspective view of an exemplary patient interface of the present disclosure comprising a nasal interface.

FIG. 2 is a perspective view of the body of the nasal interface of FIG. 1.

FIG. 3 is a schematic view of the medicament delivery system comprising the patient interface of FIG. 1.

FIG. 4 is a schematic view of an alternative configuration of the medicament delivery system of FIG. 3.

FIG. 5 is a schematic view of an alternative configuration of the medicament delivery system of FIG. 3.

FIG. 6 is a schematic view of an alternative configuration of the medicament delivery system of FIG. 3.

FIG. 7 is a schematic view of an alternative configuration of the medicament delivery system of FIG. 3.

FIG. 8 is a trend of the flow rate vs time related to the medicament delivery systems of FIGS. 3-7.

FIG. 9 is a schematic view of an alternative configuration of the medicament delivery system of FIG. 3 comprising a flow generator.

FIG. 10 is a schematic view of an alternative configuration of the medicament delivery system of FIG. 3 comprising a flow generator.

FIG. 11 is a schematic view of an alternative configuration of the medicament delivery system of FIG. 3 comprising a flow generator.

FIG. 12 is a comparative trend of the nasopharyngeal pressure vs time related to the asymmetric cannula of FIG. 1 or 2.

### DETAILED DESCRIPTION

Patient interfaces can be used for delivering breathing gases to airways of a patient. The patient interfaces may comprise nasal interfaces that can be used to deliver a flow of gases to a patient. In some configurations, nasal delivery elements, such as nasal prongs or pillows, are inserted into the nose of a patient to deliver the required therapy.

Disclosed is a system to deliver gases to a patient through a nasal interface. Preferably, the nasal interface is a non-sealed nasal interface. As described in detail below, this may be due to a leak path between at least one of the nasal delivery elements and the patient's naris. In particular one of the nasal delivery elements that substantially occludes a naris of the patient enables the creation of increase negative pressure.

In some configurations, the system provides a pressure differential at first and second nasal delivery elements of the nasal interface, with a resulting differential gases flow at the first and second nasal delivery elements. The pressure differential is provided when a patient inhales. This allows an asymmetrical or asymmetric flow to be delivered through the nasal interface to both nares. Asymmetrical flow as described herein refers to a flow that differs within the interface, within the nasal delivery elements, or within the nose. In this way, a different flow may be delivered by each nasal delivery element, or the flow may differ between inspiration and expiration, or the delivered flow may be a combination of the above. An asymmetrical flow may also include partial unidirectional flow. Delivery of asymmetrical flow may improve clearance of dead space in the upper airways, decrease peak expiratory pressure, increase safety of the therapy particularly for children and infants, and reduce resistance to flow in the interface. An asymmetrical interface, nasal delivery elements or interface includes interfaces or systems configured to produce such asymmetrical flow through asymmetrical nasal delivery elements or otherwise.

Delivery of asymmetrical flow may improve clearance of dead space in the upper airways, e.g. by flushing out CO₂ built up in the airways at the end of expiration. A nasal interface as described is configured to produce such asymmetrical flow through nasal delivery elements.

The asymmetrical flow provides a greater pressure on inhalation compared to symmetrical flow. This results in a greater inflow of gases-therefore gases flow further into the patient. This reduces leakage of gases, such as nebulised gases. It also assists with the delivery of nebulised gases into the patient.

If flow, leak, or a combination of flow and leak, is asymmetrical through the nasal interface, the flow through the nose may become asymmetrical during breathing. Partial unidirectional flow may be a type of asymmetrical flow. Partial unidirectional flow may provide improved clearance of anatomical dead space as the air is flushed from the upper airways.

Partial unidirectional flow may be more comfortable than total unidirectional flow. Total unidirectional flow herein includes all flow entering one naris by a nasal delivery element and exiting via the other naris via a nasal delivery element, venting to the atmosphere, due to the absence of a nasal delivery element, or the like. Partial unidirectional flow as described herein includes flow that may enter the nose via both nares and leave the nose from one naris, flow that may enter the nose through one naris and leave the nose via both nares, or different proportions of flow that may enter the nose through both nares and/or different proportions of flow that may leave the nose through both nares, and may be flow that may enter the nose via both nares and leave the nose from one or both nares and optionally via the mouth. If there is a pressure differential between the first and second nasal delivery elements, during inspiration the first nasal delivery element will receive more gases flow from a gases inlet than the second nasal delivery element. During expiration, the second nostril associated with the second nasal delivery element will expel more gases flow than the first nostril associated with the first nasal delivery element. The pressure differential between the first and second nasal delivery elements can change depending on whether the patient's breathing cycle is in an inspiration phase or expiration phase.

The asymmetrical flow may be applied over a suitable period. For example, the asymmetrical flow may be applied over one breath cycle of the patient or alternatively over a different number of breath cycles of the patient. The asymmetric flow results in an increased pressure on inhalation as described above. In particular, substantial negative pressure is formed in the nasal delivery element that is primarily used for inhalation. In particular a nasal delivery element that substantially occludes a naris of the patient enables the creation of increase negative pressure.

The partially unidirectional flow may reduce turbulence in the patient's nasal cavity, which could improve comfort. A reduction in turbulence can also reduce noise in the nasal cannula providing a quieter interface and improved comfort. A reduction in turbulence also reduces the likelihood that nebulised gases are deposited on the walls of the patient's naris or nares. Therefore, nebulised gases are delivered to the patient's lungs.

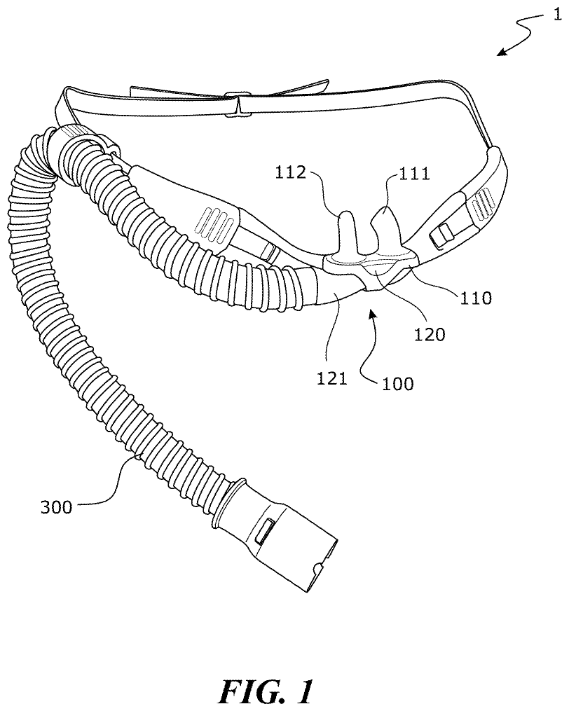

FIG. 1 shows an exemplary patient interface 1 that comprises a nasal interface 100 with nasal delivery elements comprising a first nasal delivery element 111 and a second nasal delivery element 112.

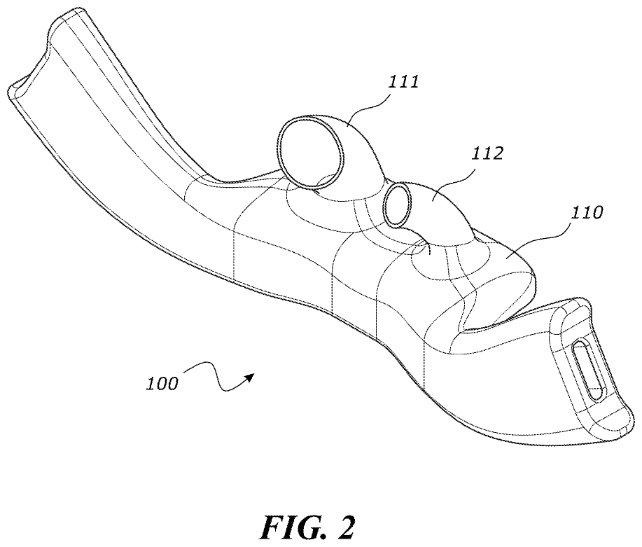

FIG. 2 shows an exemplary nasal interface 100 in more detail.

The nasal interface 100 provides a patient with a patient interface 1 suitable for the delivery of, optionally high humidity, gas flow to the patient's nasal cavity/nares. In some configurations, the nasal interface 100 is adapted to deliver a flow of gases over a wide flow range (e.g. about 8 L/min (litres per minute), or higher depending on other therapy applications, perhaps such as 10-40 L/min or 20-30 L/min). The flow rates may be bias flows averaged over time. In some configurations, the nasal interface 100 is adapted to deliver a lower flow of gases. In some arrangements, the patient interface 1 may be flow controlled.

In some arrangements the flow is dependent on pressure so it can fluctuate depending on different breathing pressures and set pressures. Wherein set pressure(s) relates to the therapy and/or patient pressure(s) which are maintained by an ancillary respiratory therapy apparatus when used in conjunction with the nasal interface of the disclosure.

The nasal interface 100 comprises a face mount part or interface body 110 part including a pair of hollow nasal delivery elements 111, 112, integrally moulded with or removably attached to the interface body 110. The nasal interface 100 comprises a gases manifold 120 part or frame 120 that comprises a gases inlet 121. The gases manifold 120 may be removably attached or integrally moulded to the respiratory conduit 300. The nasal delivery elements 111, 112 may be nasal prongs 111, 112, therefore, reference to nasal delivery elements 111, 112 herein may also be reference to nasal prongs 111, 112.

The nasal delivery elements 111, 112 may comprise inlets for the delivery of gases therethrough.

The interface body 110 part may be connectable to or engageable with the gases manifold 120 part or may be integrally formed or permanently engaged with the gases manifold 120 part. If the interface body 110 part is engageable with the gases manifold part 120, that engagement brings the first nasal delivery element 111 and the second nasal delivery element 112 into fluid communication with the gases inlet 121 such that the first nasal delivery element 111 is more proximal the gases inlet 121 and the second nasal delivery 112 element is more distal the gases inlet 121.

The interface body 110 may be formed from a soft, flexible material such as silicone, thermoplastic elastomers, or other polymers known in the art. The nasal delivery elements 111 and 112 may be supple and may be formed from a sufficiently thin layer of silicone or other suitable material to achieve this property. The interface body 110 and nasal delivery elements 111, 112 may, for example, be formed from an elastomeric material that is able to confirm to the geometry of a patient's nostril and/or cheek and provide an effective pneumatic seal.

The gases manifold 120 may be formed from a relatively harder material such as Polycarbonate, a High-Density Polyethylene (HDPE) or any other suitable plastics material known in the art. The interface body 110 provides a soft interfacing component to the patient for comfortably delivering the flow of gases through the nasal delivery elements 111 and 112, while the gases manifold 120 fluidly couples the respiratory conduit 300 to the nasal delivery elements 111 and 112 of the interface body 110.

The nasal delivery elements 111 and 112 are substantially hollow.

The first and second nasal delivery elements 111, 112 may have the same shape and configuration as each other, i.e. may be symmetrical. In other configurations, the first and second nasal delivery elements may have a different shape and/or configuration from each other, i.e. may be asymmetrical.

The interface body 110 is shaped to generally follow the contours of a patient's face around the upper lip area. The interface body 110 is moulded or pre-formed to be able to conform to and/or is pliable to adapt, accommodate and/or correspond with the contours of the user's face, in the region of the face where the nasal interface is to be located.

In the configuration shown, the interface body 110 comprises two side arms that extend laterally from either side of the interface body 110.

In some configurations, the nasal delivery elements 111, 112 extend generally upwardly and rearwardly from the interface body 110.

The gases manifold 120 is generally tubular in shape having a gases port proximal at least one side thereof, and generally extending from a front of the nasal interface 100. In some configurations, the gases port may optionally be positioned at either side thereof. At least one of the gases ports may be removably attachable to a respiratory conduit 300, such as via a threaded engagement but alternatively via a snap-fit or any other type of coupling known in the art. That enables the at least one of the gases ports to act as a gases inlet 121 for the gases manifold 120 and thereby for the nasal interface 100. Alternatively, in some configurations, the port may be fixedly coupled or integrally formed with a respiratory conduit 300.

Flow enters the nasal interface 100 through the gases port 121 and travels through the gases manifold 120 in a direction that a transverse to the direction the flow is intended to travel into the first and second nasal delivery elements 111, 112.

The gases inlet 121 is in fluid communication with the respiratory conduit 300.

In some configurations, the respiratory conduit 300 has an internal diameter of at least about 12 mm.

A gases flow path is defined by a lumen or flow channel in the gases manifold 120.

The gases manifold 120 may consist of a single part or may comprise a plurality of components that assemble together. For example, the gases manifold 120 may have a first body portion that provides the gases flow channel, and that optionally provides the gases ports and a second body portion. Alternatively, the gases manifold 120 may be a single component.

In an alternative configuration, the gases manifold 120 may comprise a single outlet, and the interface body 110 may comprise a single complementary gases entry that couples with the single outlet of the gases manifold 120 and that is in fluid communication with the first and second nasal delivery elements 111, 112 to deliver the gases to the first and second nasal delivery elements 111, 112.

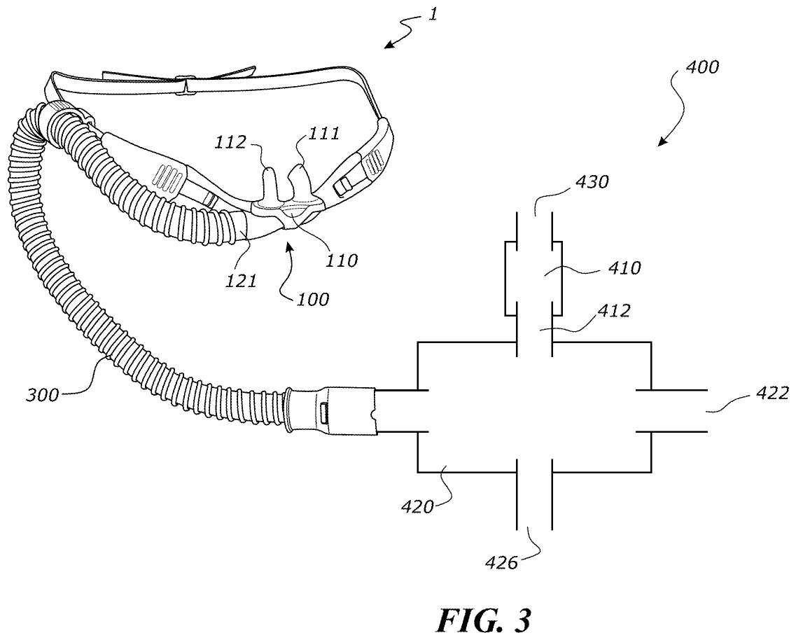

Referring to FIGS. 3-7, there is provided a medicament delivery system 400. The medicament delivery system 400 comprises a nasal interface 100 configured to deliver an asymmetric flow of gases to the nares of a patient. The nasal interface 100 may be as hereinbefore described. Therefore, the asymmetric flow may be provided by the first and second nasal delivery elements or prongs 111, 112 having a different shape and/or configuration from each other, i.e. they are asymmetrical.

The medicament delivery system 400 comprises a nebuliser 410. The nebuliser 410 converts a liquid medicine or medicament into a fine mist which can be breathed easily into the lungs.

In some arrangements, by having a nebuliser 410 as part of a system where a patient is receiving a flow of gases via a patient interface 1, there is no requirement to remove the interface to receive the nebulised medication.

Whilst reference has been made to a nebulised medication, the term nebulised gases, nebulised drugs, nebulised substance and nebulised medicament may be used to describe the same feature in some arrangements.

The nebuliser 410 comprises a medicament inlet port 412. This allows the delivery of a medicament from the nebuliser 410 through the medicament inlet port 412. The medicament inlet port 412 is in fluid communication directly or indirectly with the gases inlet 121. Therefore, the nebuliser 410 is configured to deliver a nebulised substance into the nasal interface 100.

The system defines a flow path between the medicament inlet port 412 and the nasal interface 100 via the gases inlet 121. The nebulised substance is configured to mix with ambient air to form at least part of the gases flow delivered to the nasal interface 100.

The nebuliser 410 comprises a liquid medication inlet 430 for receiving liquid medication. In some arrangements, the liquid medication is formed integral with the nebuliser 410. The nebuliser is configured to nebulise a liquid medication supplied to the nebuliser 410 via the liquid medication inlet 430 to form the nebulised substance which may be a medicament or a medication.

In some arrangements, the nebuliser 410 is vibrating mesh nebuliser 410, a jet nebuliser 410, or an ultrasonic nebuliser 410.

The asymmetric flow results in an increased pressure on inhalation as described above. In particular, substantial negative pressure is formed in the nasal delivery element 111 that is primarily used for inhalation. This may be due to the maximised total cross-sectional area of the flow path through both nasal delivery elements and minimised gases leaking between the nasal delivery element and the patient's naris, while remaining non-sealing with the patient because of the leak path between the smaller nasal delivery element and the patient's naris. In particular a nasal delivery element 111 that substantially occludes a naris of the patient enables the creation of increase negative pressure. The resultant effect is that nebulised substances are drawn further into the patient's lungs. Nebulised substances are also less likely to deposit on the passageways in the medicament delivery system 400, but also less likely to be deposited on the internal walls of the naris itself, instead being drawn into the lungs. This results in an improved uptake of medicament.

No flow generator is required to push the nebulised medication into the patient's nasal cavity. Instead, the patient's natural inhalation will generate the required flow. Negative pressure, i.e. pressure created in the respiratory conduit 300 by the patient inhaling, will be sufficient to draw the nebulised medication into the airways (nasal passages).

Referring to FIG. 3, in some embodiments, the medicament delivery system 400 comprises a medicament delivery chamber 420. The medicament delivery chamber 420 is positioned between the gases inlet 121 and the medicament inlet port 412.

The medicament delivery chamber 420 may also be referred to as a spacer 420.

Therefore, reference to a medicament delivery chamber 420 may also be reference to a spacer 420 throughout the description.

The medicament delivery chamber 420 allows a dense nebulised bolus of medicament to evenly mix with ambient air (and optionally additionally supplied oxygen), creating an inhalable gas with an optimal concentration of aerosolised particles. This also allows the dilution of the medicament. This allows the breathing at the patient's own speed to eventually consume all of the medicament that is positioned in the medicament delivery chamber 420. It also provides a passive delivery without the requirement of any complex controls for delivering medicament during breathing cycles. Therefore, there is increased compliance.

The nebulised medication does not pass through a heated tube (such as in other devices), or through temperature sensor. This means there is no issue of the nebulised medication accumulating on the interior walls of a heated tube, or the nebulised medication damaging temperature sensors. While the nebulised medication may continue to accumulate on the interior walls of the respiratory conduit 300 and/or the medicament delivery chamber 420, the interior surface area is comparatively small. Therefore, any deposition resulting in reduced delivered medication is negligible. Further, the respiratory conduit 300 is a routinely replaceable part unlike some other parts of a respiratory system. Therefore, the replacement will occur before nebulised medication deposition becomes an issue.

The medicament delivery chamber 420 is directly or indirectly in fluid communication with the gases inlet 121 and the medicament inlet port 412.

As shown in FIG. 3, the medicament delivery chamber 420 comprises an ambient air inlet 422. This allows the delivery of air to the patient, such as through inhalation. The ambient air mixes with the medicament in the medicament delivery chamber 420 forming the aforementioned bolus of medicament. Where a gas delivery apparatus is used, the respiratory conduit 300 may be removably connectable to the gas delivery apparatus. The medicament delivery system 400 is interchangeable with the gas delivery apparatus by changing the connection of the respiratory conduit 300. Therefore, when nebulising is required the medicament delivery system 400 may be connected to the respiratory conduit 300.

Referring again to FIG. 3, the medicament delivery chamber 420 is connected to the nebuliser 410 via the medicament inlet port 412. Therefore, the ambient air and medicament forms a mixture in the medicament delivery chamber 420.

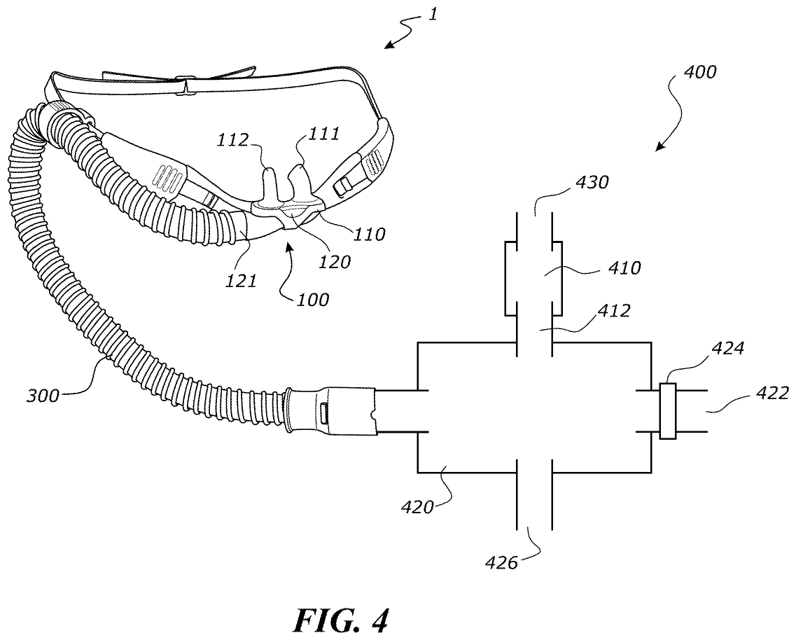

Referring to FIG. 4, a medicament delivery system 400 is shown having the same features as described with reference to FIG. 3. Therefore, the same reference numbers and descriptions apply to same features herein. Additionally, the medicament delivery system 400 of FIG. 4 comprises a one-way valve 424. The one-way valve 424 is positioned at the ambient air inlet 422. The one-way valve 424 prevents the nebulised substance flowing out of the medicament delivery chamber 420 in a direction away from the patient, for instance, through the ambient air inlet 422. Therefore, the full dose is delivered to the patient.

The one-way valve 424 also prevents air from flowing from the patient, reducing the amount of viruses and carbon dioxide from entering in through the patient end of the medicament delivery chamber 420, and preventing the patient from rebreathing exhaled air.

The one-way valve 424 may be positioned upstream of the ambient air inlet 422.

However, other arrangements are possible, such as positioning the one way valve 424 in the medicament delivery chamber 420 itself to be in a downstream position.

In arrangements without the one-way valve 424, there are benefits such as reducing breathing resistance, i.e. PIP (peak inspiratory pressure). This allows expiratory gases to pass through the system at low pressure-reducing PEEP (positive end-expiratory pressure).

Alternatively or additionally to the one-way valve 424, the medicament delivery system 400 may comprise a flow restrictor in fluid communication with the air inlet port 422. As with the one way valve 424, the flow restrictor may be positioned upstream of the air inlet port 422.

However, in some arrangements it may be in a downstream position. The flow restrictor restricts a gases flow rate through the medicament delivery system 400.

The restrictor introduces an upper limit to the total flow through the medicament delivery system 400. This may force the patient to take deeper breaths which has benefits in some situations. When the patient inhales, a percentage of air is forced to pass through the gaps in the nasal delivery elements 111, 112 to meet inspiratory demand, diluting the breathing gas.

This may be useful for diluting some medication that are unpleasant at high concentrations. A restrictor may increase breathing resistance and may encourage the patient to inhale slower.

Slower inhalation increases the amount of medication that bypasses the nasal cavity and reaches the lung.

As shown in FIGS. 3 and 4, the medicament delivery chamber comprises an oxygen intake port 426. The oxygen intake port 426 is for receiving oxygen from an oxygen source. This allows the oxygen to mix with ambient air and the medicament in the medicament delivery chamber 420 to form at least part of the gases flow and the dense bolus of nebulised medicament and gases for inhalation. In some arrangements, the oxygen source may be an oxygen tank.

The oxygen may be provided at a flow rate of approximately 15 L/min to 95 L/min, optionally 25 L/min to 80 L/min, optionally between 35 L/min and 70 L/min.

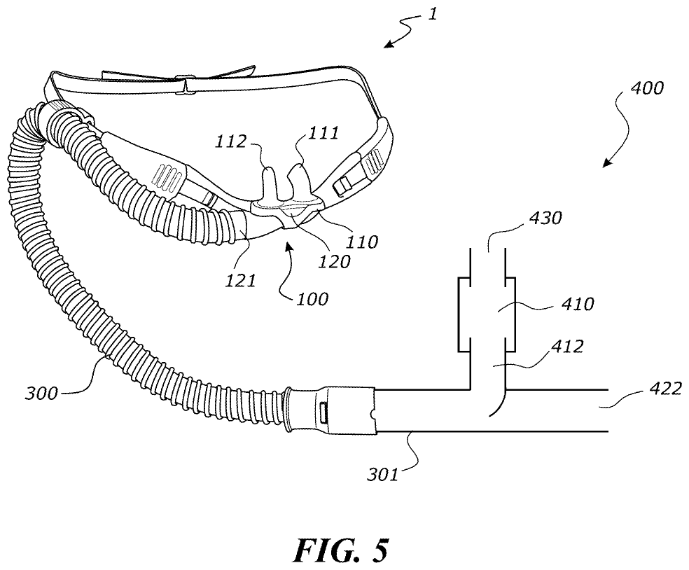

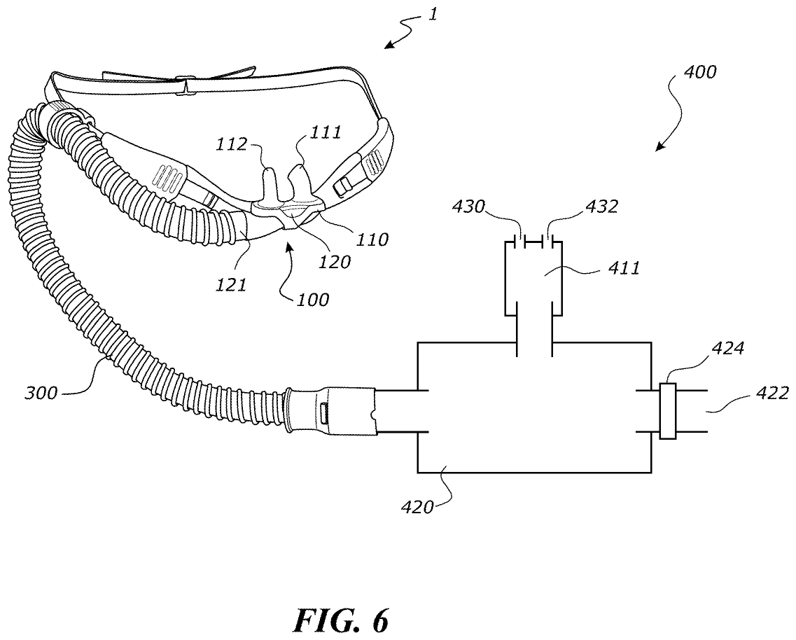

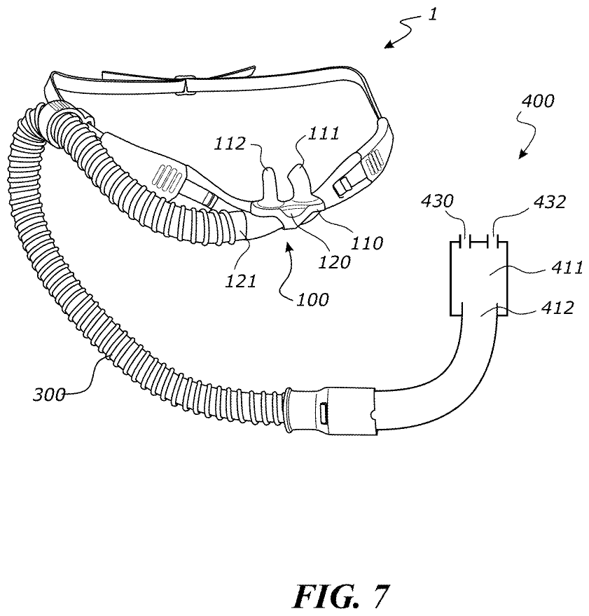

Referring to FIG. 5, a medicament delivery system 400 as described with reference to FIGS. 3 and 4 is provided where the same reference numbers and descriptions apply to same features herein. Whilst not shown, the one-way valve 424 and the restrictor described with reference to FIG. 4 may be optionally applied to the arrangement of FIG. 5 with the same advantages. Whilst no oxygen port 426 is shown, this too may be optionally provided, or an alternative source of oxygen may be provided as described with reference to FIGS. 6 and 7 described below.

The arrangement of FIG. 5 provides an inlet conduit 301. In such an arrangement the inlet conduit 301 is the medicament delivery chamber 420 as previously described. Therefore, the inlet conduit 301 is connected to the nebuliser 410 via the medicament inlet port 412.

The inlet conduit 301 may be removably attachable to the distal end of the respiratory conduit 300 or is formed as the respiratory conduit 300 itself. Therefore, the respiratory conduit 300 maybe integrally formed with an inlet conduit 301 at the end distal to the patient. In another definition, the respiratory conduit 300 comprises a connection to the medicament inlet port 412 thus forming the inlet conduit 301 as described. At an end distal to the patient of the inlet conduit 301, the inlet conduit exposed to ambient air or is connectable to a high flow machine or flow delivery device to provide the ambient air inlet 422. Therefore, the inlet conduit 301 differs from the medicament delivery chamber 420 previously described as it is a conduit rather than chamber positionable between conduits.

The medicament and air mixes in the inlet conduit 301 as hereinbefore described with reference to the medicament delivery chamber 420.

Referring to FIG. 6, a medicament delivery system 400 as described with reference to FIGS. 3 and 4 is provided where the same reference numbers and descriptions apply to same features herein. The same optional features such as the one-way valve 424 or flow restrictor may be provided. In the arrangement of FIG. 6, the nebuliser 410 is a jet nebuliser 411.

The jet nebuliser 411 comprises the liquid medication inlet 430 as previously described with reference to the nebuliser 410. Additionally, the jet nebuliser 411 also comprises a pressurised gases inlet 432. The pressurised gases inlet 432 is connected to a compressed air or compressed oxygen supply. Therefore, two inlets are provided at the jet nebuliser 411 which provide medicament and air/oxygen into the jet nebuliser 411.

The jet nebuliser 411 nebulises the medicament and this is delivered through the medicament delivery port 412 with the air/oxygen into the medicament delivery chamber 420.

The delivered medicament is then further mixed with ambient air in the medicament delivery chamber 420 as previously described.

The oxygen inlet port 426 may not be required if oxygen is provided through the pressurised gases inlet 432. However, in some arrangements, oxygen may additionally or alternatively be delivered to the medicament delivery chamber 420 as previously described.

The jet nebuliser 411 does not require a power source and is driven by compressed air or compressed oxygen. The amount of air provided through the jet nebuliser 411 may be insufficient for breathing. In such situations, such as illustrated in FIG. 6, the ambient air inlet 422 remains necessary to introduce ambient air to meet inspiratory demand. A pressure regulator may be provided in the jet nebuliser 411, such as at pressurised gases inlet 432 if the pressure of the air/oxygen is too high.

The jet nebuliser 411 provides the benefit of having a forced supply through the jet nebuliser 411 and thus the medicament delivery chamber 420. Whilst not essential, the one way valve 424 prevent the forced flow from flowing in the direction away from the patient.

Whilst not shown, in some arrangements, the air inlet 422 and medicament inlet port 412 are combined into a common port. This may also apply to the air supply for the jet nebuliser 411 where the pressurised gases inlet 432 is combined with the medicament inlet port 412.

Therefore, the nebuliser 410 may be a regular nebuliser 410 but the forced flow is provided at the medicament inlet port 412.

Referring to FIG. 7, the jet nebuliser 411 of FIG. 6 is provided with the medicament delivery system 400 where the same reference numbers and descriptions apply to same features. In the arrangement of FIG. 7, medicament inlet port 412 of the jet nebuliser 411 is connected directly to the respiratory conduit 300. Therefore, as with the arrangement of FIG. 5, the respiratory conduit 300 (or the inlet conduit portion 301 of the respiratory conduit 300) is the medicament delivery chamber 420.

In such an arrangement, there is no separate ambient air inlet 422. Therefore, if there is a greater ambient air demand than provided through the jet nebuliser 411, the medicament delivery system 400 is configured for the patient to breathe through the gaps in the patient interface 100 to meet inspiratory demand.

The respiratory conduit 300 may be removably connected between a therapy device, such as the gas delivery apparatus and the jet nebuliser 411. Therefore, when a medicament is required for delivery, the respiratory conduit 300 is swapped.

The arrangements described herein may form parts of larger systems. In some arrangements a respiratory therapy system is provided which comprises a liquid medication source for providing to the nebuliser 410 and the gas delivery apparatus configured to provide respiratory therapy to a patient. The medicament delivery system 400 is then formed as part of this. This allows the reconfiguration between the therapy delivery of the gas delivery apparatus and the medicament delivery of the medicament delivery system 400.

Such a system may be operated by controlling a flow generator of the system to generate a flow of ambient air to the medicament delivery system 400. The nebuliser 410 is also controlled to deliver nebulised medication to the medicament delivery system 400. This may be coordinated with the flow of ambient air. Therefore, the flow generator is controlled such that a gases flow of ambient air and nebulised medication is delivered to the patient via the nasal interface 100.

The respiratory therapy system may comprise further features to assist with the therapeutic functions. For instance, a blood oxygen saturation sensor, an oxygen inlet and a valve in fluid communication with the oxygen inlet to control a flow of oxygen through the oxygen inlet may be provided. A controller may be provided that is configured to control the valve based on at least one measurement of oxygen saturation from the blood oxygen saturation sensor.

The respiratory therapy system described may be operated to provide nebulised medication. When providing nebulised medication, the nebuliser 410 and/or spacer 420 may be directly or indirectly in fluid communication between the liquid medication source (i.e. inlet 430) and the nasal interface 100. The nebulised medication is delivered to the nasal interface 100 from the liquid medication source. The nebulised medication is drawn into the patient's nares by the asymmetric flow at the nares of the patient.

The nebuliser 410 and/or spacer 420 may be connected to the gases inlet 121 or gases inlet tube (conduit 300) of the nasal interface 100.

As described herein, a non-sealing nasal interface 100 that delivers an asymmetric flow of gases to the nares of a patient creates substantial negative pressure. In particular, substantial negative pressure is formed in the nasal delivery element that is primarily used for inhalation.

The nasal delivery element that substantially occludes a naris of the patient (i.e. the larger cross-section nasal delivery element) enables the creation of increase negative pressure. The resultant effect is the improved draw of nebulised substances into the patient's lungs.

The increased pressure formed by the asymmetric nasal interface 100 also allows the entrainment of ambient air. This ensures that ensuring inspiratory demand is met for a patient even when any flow generation is not functioning/present. This is especially the case when either nebulised drugs are being delivered, or when the nebuliser/spacer 420 is directly connected to the nasal interface 100.

As discussed, advantages of asymmetric nasal prongs 111, 112 includes one of the prongs providing more occlusion of naris which generates a larger negative pressure during inhalation, whilst a smaller prong provides a leak path that permits comfortable exhalation through the gaps around the prong. The configuration allows the patient to breathe comfortably through the nose while keeping the interface 100 in place after the session, which may be particularly advantageous for patients who are unable to easily remove the interface 100. The asymmetric cannula also allows the patient to receive nebulised drugs without a flow source.

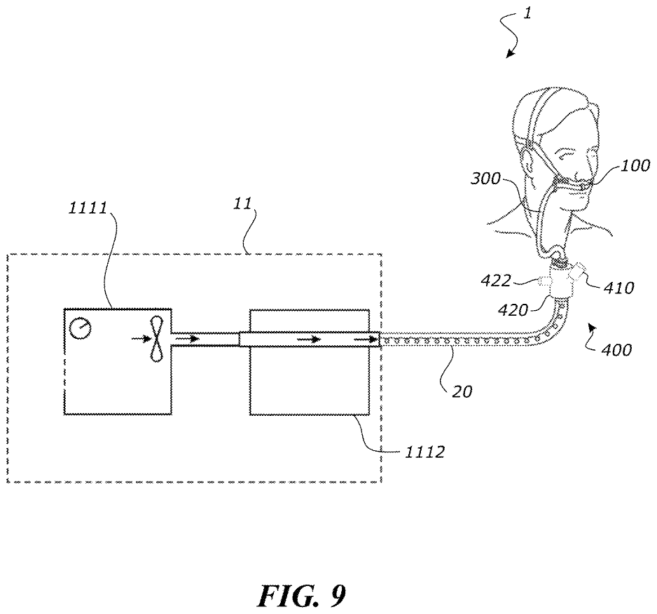

The respiratory therapy system described provides respiratory therapy and nebulised medication to the patient. The system is switchable between providing respiratory therapy and nebulised medication. In one configuration, a flow generator (11—described in more detail below with reference to FIGS. 9 to 11) is connected to the gases inlet 121 or gases inlet tube (conduit 300) of the nasal interface 100. The flow generator 11 is controlled to deliver a flow of ambient air and/or oxygen to the nasal interface 100. In a further configuration the nebuliser 410 and/or spacer 420 may be directly or indirectly in fluid communication between the liquid medication source (i.e. inlet 430) and the nasal interface 100. The nebuliser 410 and/or spacer 420 may be connected to the gases inlet 121 or gases inlet tube (conduit 300) of the nasal interface 100. The nebulised medication is drawn into the patient's nares by the asymmetric flow at the nares of the patient.

As described above, the increased pressure formed by the asymmetric nasal interface 100 ensures that inspiratory demand is met for a patient even when flow generation is not operating/present.

In switching between the configurations, the flow generator 11 is switched off and/or disconnected. However, as described further below, in some alternative arrangements, the flow generator 11 remains switched on and/or connected. However, the nebulised medication remains drawn into the patient's nares by the asymmetric flow at the nares of the patient.

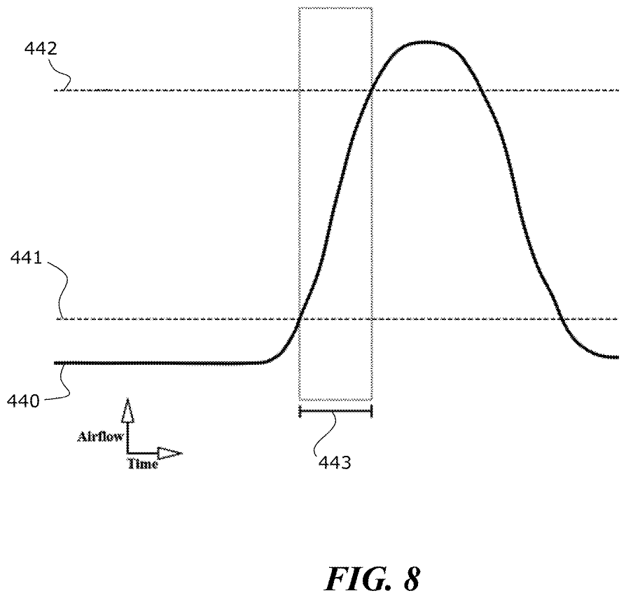

Referring to FIG. 8, a medicament delivery system 400 as hereinbefore described, such as with reference to FIGS. 3-7 is provided. The medicament delivery system 400 is breath actuated. Therefore, medicament delivery system 400 automatically detects when the patient is inhaling, and upon this detection, the nebuliser 410 is activated to dispense the nebulised medication into the airflow.

FIG. 8 shows a trend 440 of airflow vs time. The airflow is measured as patient inhalation. This may be measured by a flow sensor continuously monitoring the amount of flow being generated by the inhalation.

The nebuliser 410 may be activated at the beginning of inhalation such as at a lower activation threshold 441 when sufficient flow is detected. This may be a percentage of maximum flow, such as between 0% and 10% of maximum flow rate, or once the flow rate reaches a minimum percentage of maximum flow such as above 5% or above 10%. As gases inhaled at the beginning of a cycle reach the lungs, it is beneficial that activation occurs as soon as possible when inhalation begins. However, it is also beneficial to avoid erroneously activating the nebuliser 410 prematurely such as during a short breath. The nebuliser 410 may be deactivated when the differential of airflow has dropped below a certain threshold. Alternatively, as illustrated in FIG. 8, the nebuliser may be deactivated at an upper activation threshold 442. Therefore, even though the airflow continues to increase, the nebuliser 410 is no longer active.

Such an approach maximises efficiency and minimise drug wastage as nebulised medication is only introduced to the portion of gas that reaches the lung. A portion of inhalation volume never reaches the lungs. For instance, it is estimated that the anatomical dead space in the respiratory system is 150 ml, and that the 150 ml of gas inhaled at the end of the inhalation never reaches the lungs. Therefore, an upper activation line may be between 100 ml and 200 ml of the volume for the maximum airflow. This is calculated with reference to an activation time 443 between the lower 441 and upper 442 activation thresholds.

The activation maybe binary, where the nebuliser 410 is either in the on or off state.

Alternatively, the activation may be continuous, where a flow sensor continuously monitors the amount of flow being generated by the inhalation, then accordingly adjusts the rate at which the nebulised medication is dispensed by nebuliser 410 throughout the breathing cycle.

Referring to FIG. 9, there is provided a medicament delivery system 400 as previously described with reference to any of FIGS. 3-7. The medicament delivery system 400 of FIG. 9 is shown to comprise the medicament delivery chamber 420. However, in some alternative arrangements the medicament delivery chamber 420 may be omitted, such as shown in FIG. 7. Likewise, whilst an ambient air inlet 422 is shown, this too may be omitted in some arrangements.

In the illustrated arrangement, the medicament delivery system 400 comprises the nebuliser 410. However, this may a jet nebuliser 411 in some arrangements. The medicament delivery chamber 420 comprises the (optional) ambient air inlet 422. The medicament delivery chamber is positioned at an end of the conduit 300 which connects to the nasal interface 100.

As previously described, the nasal interface 100 may be configured to deliver an asymmetric flow of gases to the nares of a patient.

The conduit 300 may have a relatively short length. Alternatively, the medicament delivery chamber 420 (or nebuliser 410) may be proximal or directly connected to the nasal interface 100.

Where the amount tubing (i.e. conduit 300) between the nebuliser 410 and the patient is minimised, this reduces the area in which nebulised medication may accumulate, e.g. on the inside of the tubing. A shorter conduit 300 reduces loss of medication and increases the delivered dose.

In some arrangements, the length of the tubing (e.g. conduit 300) is less than 1000 mm. In other arrangements, the length of the conduit is less than 700 mm.

A further benefit of a shorter or removal of a conduit 300 is to minimise the phase delay between detection of patient inhalation and delivery of medicament to the nares. Inhalation detection has been described both above and below. This shorter distance can be particularly important for hyperventilated patients who breathe at a higher rate with a smaller tidal volume. If phase delay is too long, the medicament may be delivered later in the breath cycle than intended, or the patient may need to inhale multiple times before receiving the medicament.

As previously described, a forced supply, such as a flow generator 11 may be connected to the medicament delivery system 400, such as to the medicament delivery chamber 420 (via the oxygen intake port 426) or nebuliser 410, or to the ambient air intake port 422. This can help drive the nebulised flow. The flow generator 11 may be a high flow machine 11 to produce a high flow.

The flow generator 11 may comprises a blower 1111 that controls flows delivered to a patient via a supply conduit 20 and patient interface 1. The supply conduit 20 connects the flow generator 11 to the patient interface conduit 300. Where a medicament delivery system 400 is present, the medicament delivery chamber 420 or nebuliser 410 may be positioned between the supply conduit 20 and the patient interface conduit 300. In some arrangements, the supply conduit 20 and the nasal interface conduit 300 are the same conduit, and may be continuous.

The blower 1111 may be a centrifugal blower, comprising at least a motor and impeller or fan that is driver by the motor. Other types of blowers may be employed, such as axial blowers. The flow rate and/or pressure of flows of gases being output by the flow generator 11 can be controlled by varying the output of the blower 1111, for example by varying the rotational speed of the motor driving the impeller or fan. The flow generator may be configured to provide flows of gases at high flow rates. Examples of high flow rates are provided later.

The flow generator 11 may optionally comprise a humidifier and/or sensor module 1112. An inlet of the sensor module 1112 may be fluidically/pneumatically connected to the outlet of the blower 1111, or may instead be positioned upstream of the blower in other embodiments.

The sensor module 1112 may be located prior to humidifier part of the combined module 1112.

One or more sensors (for example, Hall Effect sensors) may be used to measure a motor speed of the blower motor.

Positioning sensors (e.g., flow rate, pressure, oxygen fraction, and/or other types of sensors in the sensor module 1112) downstream of the blower 1111 can increase accuracy of measurements, such as the measurement of fractional gas concentrations, including oxygen fraction, over systems that position the sensors upstream of the blower and/or a mixer.

Positioning these sensors at a location further along the flow pathway, after the flow of gases has been more mixed (and may therefore be more homogeneous), may yield more consistent and/or repeatable measurements.

The humidifier part of the module 1112 may be provided between the blower 1111 and the supply conduit 20 to humidify the flow of gases being output by the flow generator 11.

Humidification is particularly useful when providing high flow therapy (where high flow rates of otherwise dry gases are delivered to the patient's airways) as it improves the tolerability and comfortability of the therapy. Increasing the humidity of the gases to or closer to the natural levels in a healthy patient's airways may help to maintain the condition of the airways, reducing or preventing drying-out or other effects which may cause discomfort and adverse health outcomes. In some configurations the humidifier part of the module 1112 may be optional, in which case the apparatus may provide non-humidified gases from the flow generator 11 to the patient.

The humidifier 1112 may be a heated humidifier, wherein the humidifier comprises at least one heating element. The humidifier 1112 may be a heated pass-over humidifier.

A controller (not shown) of the flow generator 11 (e.g. high flow machine) receives data indicative of flow rate from a flow rate sensor, such as the sensor module 1112 in the air path of the flow generator 11. The controller is connected to the nebuliser 410 and can control activation of the nebuliser 410 of the medicament delivery system 400.

The controller may operate in different modes, the modes may comprise a therapy mode and a nebuliser mode.

In the nebuliser mode the flow generator 11 may be turned off or remain on but set at a low flow rate. The humidifier module 1112 of the high flow machine may be turned off or remain on. Turning the humidifier module 1112 off would be beneficial in preventing humidity from diluting the nebulised substances. The humidifier module 1112 remaining on is beneficial in avoiding re-warming the medicament delivery system 400 after returning to therapy mode, and to provide some humidity for any ambient air pulled through the ambient air inlet. If the humidifier module 1112 remains on, it is preferred for the flow generator 11 to remain on at low flow rates (1-2 L/min) to reduce condensation.

The controller of the flow generator 11 receives the flow rate from a flow rate sensor (such as in the sensor module 1112) to detect patient inhalation. Upon detection of inhalation, the controller sends an activation signal to the nebuliser 410 to nebulise the medicament. The controller may operate according to the flow rate profile described in reference to FIG. 8 above.

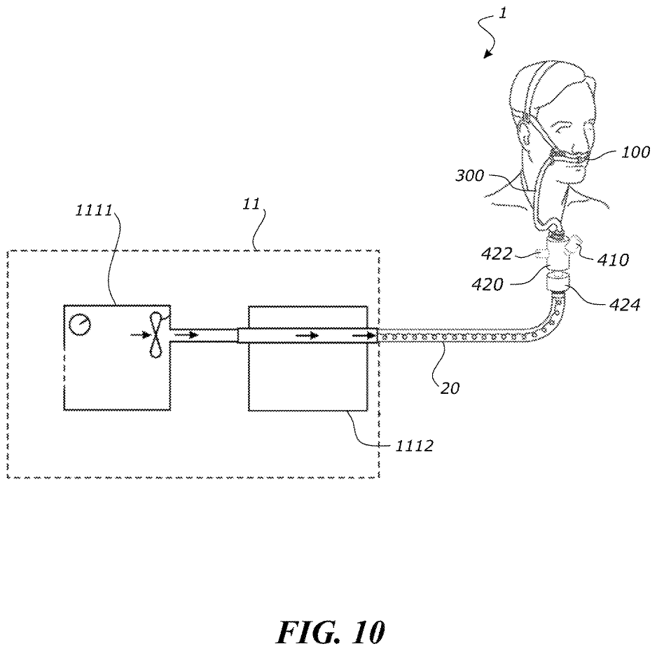

Referring to FIG. 10, there is provided a medicament delivery system 400 as previously described with reference to FIG. 9. Therefore, the description for FIG. 9 likewise applies for FIG. 10. Additionally, in the illustrated arrangement, the medicament delivery system 400 comprises a one-way valve 424. The one-way valve 424 may be as described above, such as with reference to FIG. 6. Whilst the one-way valve of FIG. 10 is shown to be positioned between a forced air supply (flow generator 11) and the medicament delivery chamber 420, in alternative arrangements, the one-way valve 424 may be positioned on an ambient air inlet 422.

In some arrangements, the one-way valve 424 may be positioned in the supply conduit 20. The position may be proximal to the flow generator 11 or may be proximal to the patient. In other arrangements, the one-way valve 424 may be positioned in the flow generator 11, such as near an outlet to the supply conduit 20.

An example function of the one-way valve 424 is as follows, when the patient inhales, the one-way valve 424 opens and gases are delivered from the flow generator 11. When the patient exhales, the one-way valve 424 closes and gases exit through the patient interface 100, e.g. through gaps such as around the prongs, where applicable.

In an alternative arrangement, the controller of the flow generator 11 may be operated to provide selective flow. Such an arrangement may be alternative to a one-way valve 424, and thus may be applicable to the arrangement of FIG. 9. In other arrangements, the selective flow may be additional to a one-way valve 424 such as shown in FIG. 10.

In the arrangement, the controller of the flow generator 11 (in a nebuliser mode) may control the blower 1111. During patient exhalation, the blower 1111 is controlled to oppose reverse flow so as to maintain a substantially zero net flow toward the nebuliser 410. During patient inhalation, the blower 1111 is either inactive or is operated to assist the patient's inhalation, thereby permitting forward flow toward the patient. In some arrangements, when operating, the flow rate generated by the blower 1111 is low, e.g. less than about 10 L/min, or preferably less than about 5 L/min, or preferably less than about 3 L/min. The low positive flow rate reduces condensation forming in the gases pathway (e.g. supply conduit 20 and/or conduit 300) and/or reduces humid air flow back into the blower 1111 and condensing in the blower 1111.

A single nebulisation session may typically last between 10 and 20 minutes. Typically, the duration of a nebulisation session provides about 5-6 ml of medicament. Following completion of the session, the patient may continue to wear the nasal interface 100. An advantage of the asymmetric nasal prongs is that the larger prong provides more occlusion of nostril which generates a larger negative pressure during inhalation, while the smaller prong provides a leak path that permits comfortable exhalation through the gaps around the prong.

The configuration allows the patient to breathe comfortably through the nose while keeping the interface 100 in place after the session, which may be particularly advantageous for patients who are unable to easily remove the interface. The asymmetric cannula also allows the patient to receive nebulised drugs without a flow source.

Efficiency of medicament delivery improves when the flow rate is low. Traditional high-flow machines deliver breathing gas at a high flow rate. It is generally counter-intuitive to turn off the flow generator 11 to improve delivery efficiency. This may be because conventional high flow therapy employs symmetric, non-sealing nasal prongs that do not generate sufficient negative pressure during patient inhalation to reliably draw medicament into the airways without an active flow source. The asymmetric nasal prongs of the present disclosure overcome this limitation by generating sufficient negative pressure during inhalation, thereby enabling effective medicament delivery with the flow generator 11 turned off.

Optionally, in some arrangements, the flow generator 11 may supply 0.5-5 L/min of oxygen during medicament delivery for patients who require supplemental oxygen. Alternatively, in some alternative arrangements, the supplemental oxygen may be connected directly to the medicament delivery chamber 420. The flow generator 11 may be switched off during oxygen delivery.

In another alternative arrangement, a single-prong nasal cannula 100 is used in place of an asymmetric nasal cannula. All functional benefits described above may be similarly achieved using a single-prong cannula.

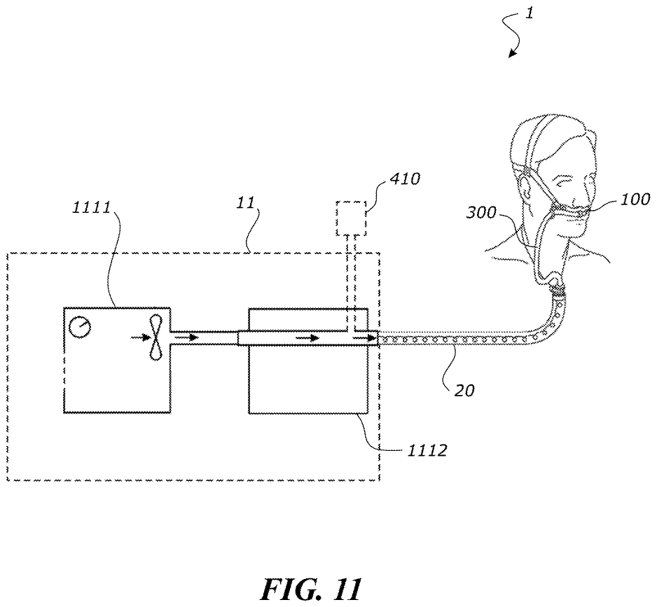

Referring to FIG. 11, there is provided a medicament delivery system 400 as previously described with reference to FIG. 9. Therefore, the description for FIG. 9 likewise applies for FIG. 10. In the illustrated arrangement of FIG. 11, the medicament delivery system 400 is part of the flow generator 11. The medicament delivery system 400 may comprise a nebuliser 410 that provides nebulised medicament near an outlet of the flow generator 11.

Optionally, a medicament delivery chamber 420 may be provided as part of the medicament delivery system 400. Such an arrangement may provide additional mixing with the air supply, such as when it travels through the length of the supply conduit 20. As with the arrangements described above (and examples provided below), the asymmetric nasal interface of the present disclosure generates sufficient negative pressure during inhalation to draw the nebulised drugs into the patient's airways (i.e. without a flow generator 11).

Alternatively, the one-way valve may be positioned close to the interface, as illustrated above in FIG. 9.

In some arrangements, such as in the arrangements of FIGS. 9 to 11, but also in the arrangements of FIGS. 3 to 7 when a flow generator 11 is provided, the flow generator 11 may be controlled to provide a flow of gases while the nebuliser 410 is operating. Such an arrangement may include any of the nebuliser systems above, such as medicament delivery chamber 420. The flow generator 11 and nebuliser are co-connected (i.e. the devices are not disconnected to provide the various operations). The flow from the flow generator 11 may provide high flow. The high flow may optionally be humidified, such as via the humidifier 1112.

A humidified high flow is as described above. One non-limiting example of a humidified high-flow delivery of gases is the providing of humidified gases at or above 15 L/min for adults or 2 L/min per kg for infants.

The asymmetric flow due to the asymmetric nasal interface 100, i.e. via the asymmetric prongs helps to draw gases deeper or further into the patient's airways, such as by the negative pressure discussed herein causing an effective suction. The use of a nebuliser with the asymmetric nasal interface 100 allows for nebulised medication to move further or deeper into the nares of the user and for the medicament to deposited deeper in the airways. The asymmetric flow through the asymmetric prongs improves nebulised medication delivery. In some instances, the asymmetric flows allows an increased amount of drugs to be deposited into the lungs of the patient.

Operation of the flow generator 11 to provide high flow helps to push gases toward the patient. Further, the high flow may also assist with the delivery of gases (e.g. respiratory gases) deeper into a patient's airways.

The combined delivery of nebulised medication and operation of the flow generator 11 to provide high flow helps to push nebulised medication toward the patient and provide the nebulised medication further into a patient's airways. Therefore, an overall improvement in the delivery of nebulised medication is provided.

When the nebuliser is not operating, the high flow may be provided by the flow generator 11.

Whilst, with reference to FIG. 8, a detection for inhalation is described, in some arrangements which combine a flow generator 11 and a nebuliser 410 co-connected, the flow generator 11 may be operated to reduce flow rate (e.g. from a high flow). The reduction of the flowrate of the flow generator 11 may be when nebulisation operation is detected. The reduction of the flowrate of the flow generator 11 may be when nebulisation operation is activated.

Activation of the nebulisation operation may be via a user interface. A user interface may be a screen/display on the patient interface 1 or may be via an application such as on a mobile device.

The reduction of flowrate of the flow generator 11 during the nebulising operation may help to reduce nebulised medication from being deposited in the supply conduit 20 or conduit 300. The reduction in flowrate may also prevent nebulised medication crashing into the supply conduit 20 or respiratory conduit 300, this may again deposit the medicament on the conduits. The reduction of flowrate of the flow generator 11 may include the stopping of flow. However, in some arrangements, a low flow may ensure that nebulised medication remains pushed toward the patient and away from the blower 1111.

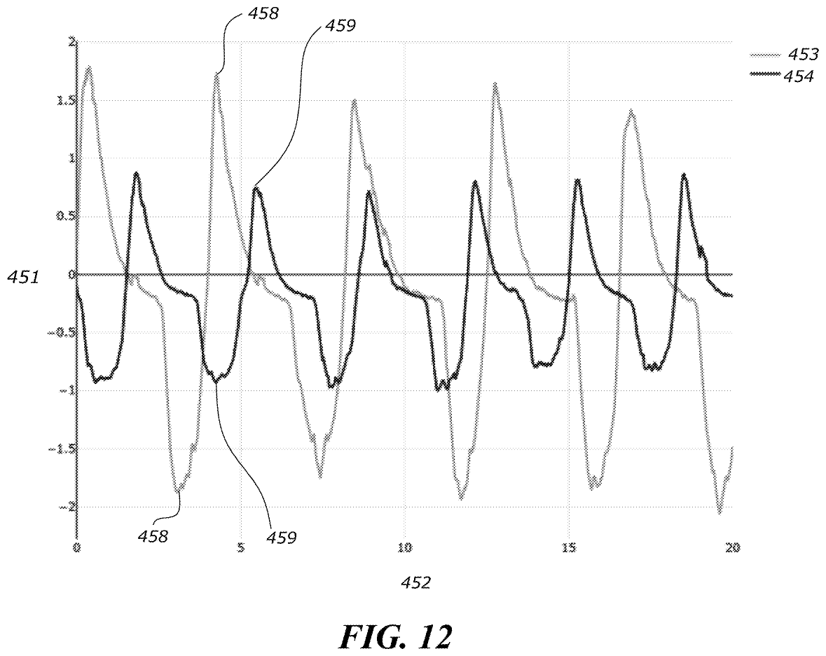

Referring to FIG. 12, a trend is provided comparative test results between an asymmetric cannula and a symmetric cannula.

On the y-axis 451 is the nasopharyngeal pressure measured in cmH₂O. On the x-axis 452 is the time measured in seconds.

The lighter line 453 is the trend for the asymmetric cannula. The darker line 454 is the trend for the symmetric cannula.

The comparative testing was conducted to evaluate the amount of negative pressure generated by an asymmetric cannula 453 relative to a symmetric cannula 454. In the test setup, a pressure sensor was connected to the open end of the tube, and the interface was donned by a person. The person then breathed normally. The pressure sensor measured pressure within the tube of the interface during normal breathing.

As shown in the trend, the negative pressure generated during inhalation was significantly greater when using the asymmetric cannula as shown by points 456, 458 compared to the symmetric cannula as shown by points 457, 459 on the trend.

The magnitude of the positive nasopharyngeal pressure 451 for the asymmetric cannula 453 as shown by point 456 is approximately 1.5 to 1.8 cmH₂O on each respiratory or breath cycle. Comparatively, magnitude of the positive nasopharyngeal pressure 451 for the symmetric cannula 454 as shown by point 457 is approximately 0.7 to 0.8 cmH₂O on each respiratory or breath cycle. There is an approximate increase in positive pressure of about 1 cmH₂O for the asymmetric cannula compared to the symmetric cannula.

The magnitude of the negative nasopharyngeal pressure 451 for the asymmetric cannula 453 as shown by point 458 is approximately −1.7 to −2.0 cmH₂O on each respiratory or breath cycle. Comparatively, magnitude of the positive nasopharyngeal pressure 451 for the symmetric cannula 454 as shown by point 459 is approximately −0.8 to −1.0 cmH₂O on each respiratory or breath cycle. There is an approximate increase in negative pressure of about 1 cmH₂O for the asymmetric cannula compared to the symmetric cannula.

The positive nasopharyngeal pressure 451 is on an exhalation (or expiration) phase of a respiratory or breath cycle and the negative nasopharyngeal pressure 451 is on an inhalation (or inspiration) phase of a respiratory or breath cycle.

The magnitude of the negative pressure 451 is sufficient to draw gases through a nebuliser 410 without the use of a separate flow generator 11.

As shown in the experimental results of FIG. 12, the asymmetric nasal interface 100 results in an increased pressure on inhalation as described above. In particular, substantial negative pressure is formed in the nasal delivery element that is primarily used for inhalation.

The nasal delivery element that substantially occludes a naris of the patient (i.e. the larger cross-section nasal delivery element) enables the creation of increase negative pressure. The resultant effect is the improved draw of nebulised substances into the patient's lungs.

The increased pressure formed by the asymmetric nasal interface 100 also allows the entrainment of ambient air. This ensures that ensuring inspiratory demand is met for a patient even when any flow generation is not functioning/present. This is especially the case when either nebulised drugs are being delivered, or when the nebuliser is directly connected to the nasal interface, such as in the various arrangements shown above.

As shown in FIG. 2, in some arrangements to provide the asymmetric flow, the left nasal delivery element 111 is larger than the right nasal delivery element 112. Specifically, the left nasal delivery element 111 has a larger cross-sectional area (taken along the same plane) than the right nasal delivery element 112. The total area of the patient's nares that is blocked is at least 50%.

It will be appreciated that in some arrangements the right nasal delivery element 112 may be larger than the left nasal delivery element 111 to provide an asymmetric flow.

In some arrangements, the larger nasal delivery element 111 substantially obstructs one of the patient's nares and can seal or substantially seal with that naris, while the smaller nasal delivery element 112 obstructs the other naris to a lesser extent and provides a leak path between the smaller nasal delivery element 112 and the patient's naris.

When appropriately fitted to the patient, the asymmetric cannula maximises the total cross-sectional area of the flow path through both nasal delivery elements 111, 112 and minimises gases leaking between the nasal delivery elements 111, 112 and the patient's naris, while remaining non-sealing with the patient because of the leak path between the smaller nasal delivery element 112 and the patient's naris. This allows generation of substantial negative pressure in the cannula when the patient inhales, while remaining non-sealing. This provides a non-sealed nasal interface/cannula 100. The non-sealing nasal interface also provides additional safety from over pressurisation.

But Inspiratory gases travel through the nasal cavity associated with the larger nasal delivery element 111, and expiratory gases travel through the nasal cavity associated with the smaller nasal delivery element 112. This effectively eliminates the rebreathing “dead space” in both cavities. With the larger nasal delivery element 111, inspiratory gases flow through the associated naris at a lower velocity, encouraging the flow to remain laminar, be less turbulent, and reduce its tendency to form vortices. These four factors reduce the amount of particles that impact the walls of the nasal cavity and accumulate as liquid on the walls of the nasal cavity. In other words, more nebulised medication passes through the nasal cavity and enter the lungs where the medication is most effectively absorbed into the bloodstream. Therefore, the medicament delivery system 400 is more effective with the asymmetric nasal interface 100.

In view of the above, the asymmetric nasal interface 100 provides the advantages of both large cannulas and small cannulas. Large cannulas are advantageous for allowing the patient to draw nebulised gases by inhalation without a flow generator, due to higher occlusion of nares and elevated resistance, which creates negative pressure during inspiration and leads to inhalation of the nebulised substances. Small cannulas are advantageous for being able to deliver a high flow of therapy gas, without over-pressurising the nasal cavity. Small cannulas also reduce inspiratory and expiratory resistance. Further, the non-sealing nasal interface is improved over sealed masks by: reduced rebreathing, because of less dead space; inspiratory pressure demand can be met by the permittance of ambient air through the gaps between the prongs and the nares, effectively reducing PIP. With a sealed mask, the patient can be left gasping for air when they attempt to inhale at a flow rate higher than what the flow generator can permit; ability for the patient to switch between nasal high flow and medication delivery without taking the nasal interface off. This may be useful for patients with disabilities that limit their ability to don and remove the interface.

The patient interface 1 and/or nasal interface 100 may have any one or more of the features and functionality described in PCT publication no. WO 2023/067558 or U.S. patent application Ser. No. 18/702,459. The contents of those specifications are incorporated herein in their entirety by way of reference. The features that provide or contribute to providing the asymmetric flow and parameters of the asymmetric flow may be any of the features and parameters described in those specifications.

The nasal interface 100 provides an asymmetric flow in some configurations. In use of the nasal interface in such configurations, there is a net flow from the first naris of the patient to the second naris of the patient throughout a respiratory or breath cycle.

In some configurations, the patient may be spontaneously breathing.

The breath cycle could be described to have an inspiration phase, an inflection phase where the patient is neither inspiring or expiring (this phase could also be known as a breathing holding phase), and an expiration phase. The inflection phase may occur over a significantly shorter time period than the inspiration and/or expiration phase.

Patient interfaces with nasal interfaces 100 according to the configurations described herein may be employed in a method of delivering gas to the airway of a patient in need thereof, improving the ventilation of a patient in need thereof, reducing the volume of anatomical dead space within the volume of the airway of a patient in need thereof, and/or treating a respiratory condition in a patient in need thereof, as described above.

The patient population that can most benefit from the use of the nasal interfaces 100 and patient interfaces 1 of the present disclosure are COPD or bronchiectasis patients, in particular patients suffering from overlap syndrome of OSA and COPD.

The nasal interfaces 100 and patient interfaces 1 are advantageous as they can provide pressure and asymmetric flow to flush airways. Additionally, the nasal interfaces 100 and patient interfaces provide a more comfortable and easier to use interface.

Patient interfaces comprising nasal interfaces 100 of the type disclosed herein may be used in a respiratory therapy system for delivering gases to a patient.

The asymmetric flow of gases at the patient's nasal airways may be created during an inhalation phase of the respiratory cycle. Additionally, this may also occur during an exhalation phase of the respiratory cycle. The inhalation phase and exhalation phase may define a respiratory cycle. As such, an asymmetric flow may be provided at the nasal airways of the patient by the nasal interface 100 throughout a respiratory cycle of a patient.

The nasal interfaces 100 disclosed herein could be used in a medical care facility, home environment, emergency vehicle, or any other suitable environment. Therefore, references herein to “patient” should be interpreted to be any suitable subject that the nasal interfaces are used for or by.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Features from any of the described embodiments may be combined with each other and/or an apparatus may comprise one, more, or all of the features of the above-described embodiments. Moreover, not all of the features, aspects and advantages are necessarily required to practice the present disclosure. Accordingly, the scope of the present disclosure is intended to be defined only by the claims that follow.

1-61. (canceled)

62. A medicament delivery system comprising:
a non-sealing nasal interface configured to deliver an asymmetric flow of gases to nares of a patient, the non-sealing nasal interface comprising a gases inlet;
a nebuliser comprising a medicament inlet port and in fluid communication directly or indirectly with the gases inlet, the nebuliser configured to deliver a nebulised substance into the non-sealing nasal interface;
the medicament delivery system defining a flow path between the medicament inlet port and the non-sealing nasal interface via the gases inlet,
wherein the nebulised substance is configured to mix with ambient air to form at least part of the asymmetric flow of gases delivered to the non-sealing nasal interface.

63. The medicament delivery system of claim 62, comprising a medicament delivery chamber positioned between and directly or indirectly in fluid communication with the gases inlet and the medicament inlet port.

64. The medicament delivery system of claim 63, wherein the medicament delivery chamber comprises an inlet for the ambient air and the medicament inlet port.

65. The medicament delivery system of claim 64, comprising a one-way valve upstream of an ambient air inlet to prevent the nebulised substance flowing in a direction through the ambient air inlet.

66. The medicament delivery system of claim 63, wherein the medicament delivery chamber comprises an oxygen intake port for receiving oxygen from an oxygen source, the oxygen configured to mix with ambient air in the medicament delivery chamber to form at least part of the asymmetric flow of gases.

67. The medicament delivery system of claim 63, wherein the medicament delivery system comprises a flow delivery device in fluid communication with an air inlet and positioned upstream of the air inlet to drive the gases flow in a flow path direction toward the non-sealing nasal interface to increase a gases flow rate.

68. The medicament delivery system of claim 63, wherein nasal interface comprises an inlet conduit, wherein the inlet conduit is the medicament delivery chamber.

69. The medicament delivery system of claim 63, wherein an air inlet and medicament inlet port are combined into a common port.

70. The medicament delivery system of a claim 62, wherein a stopping of delivery or a reduction of delivery rate of the nebulised substance is at a final 150 ml of an inhalation.

71. The medicament delivery system of claim 62, wherein the nebuliser is configured to nebulise a liquid medication supplied to the nebuliser to form the nebulised substance.

72. The medicament delivery system of claim 62, wherein the asymmetric flow is configured to increase an inhalation pressure formed in the non-sealing nasal interface to deliver the nebulised substance mixed with ambient air to the patient.

73. The medicament delivery system of claim 62, wherein the non-sealing nasal interface comprises first and second nasal delivery elements configured to deliver the asymmetric flow of gases to the nares.

74. The medicament delivery system of claim 73, wherein the first nasal delivery element and second nasal delivery element are asymmetric.

75. The medicament delivery system of claim 73, wherein the first nasal delivery element occludes a first naris more than the second nasal delivery element occludes a second naris.

76. The medicament delivery system of claim 73, wherein the first nasal delivery element is larger than the second nasal delivery element.

77. The medicament delivery system of claim 73, wherein the first nasal delivery element has a larger internal diameter and/or internal cross-sectional area and a larger circumference or external diameter than the second nasal delivery element at or adjacent an outlet of the first nasal delivery element.

78. The medicament delivery system of claim 73, wherein the first nasal delivery element is relatively larger and the second nasal delivery element is relatively smaller to provide a leak path for expired gases at another of the nares.

79. A respiratory therapy system comprising:
a liquid medication source;
a gas delivery apparatus configured to provide respiratory therapy to a patient, and
a medicament delivery system comprising:
a non-sealing nasal interface configured to deliver an asymmetric flow of gases a patient's nares, the non-sealing nasal interface comprising a gases inlet; a nebuliser and/or a medicant delivery chamber directly or indirectly in fluid communication with the liquid medication source and the gases inlet, the nebuliser configured to nebulise liquid medication from the liquid medication source and deliver nebulised medication to the non-sealing nasal interface, wherein the nebulised medication is configured to form at least part of a gases flow delivered to the non-sealing nasal interface.

80. The respiratory therapy system of claim 79, wherein the gas delivery apparatus comprises a blower and a humidifier, the humidifier positioned downstream of the blower and the gas delivery apparatus configured to provide humidified high flow therapy.

81. A medicament delivery system comprising:
a non-sealing interface comprising a first nasal delivery element and a second nasal delivery element, wherein the first nasal delivery element is larger than the second nasal delivery element such that the first nasal delivery element and the second nasal delivery element are configured to deliver an asymmetric flow of gases to a patient's nares;
a conduit having a length of less than 1000 mm and in fluid communication with the non-sealing interface; and
nebuliser in fluid communication with the conduit.
